(19) United States
(12) Patent Application Publication
(10) Pub. No.: US 2013/0165718 A1
(43) Pub. Date: Jun. 27, 2013

(54) PROCESS FOR THE PREPARATION OF OLEFINS

(71) Applicant: SHELL OIL COMPANY, Houston, TX (US)

(72) Inventors: Frederik Hendrik VAN DER STEEN, Amsterdam (NL); Jeroen VAN WESTRENEN, Amsterdam (NL)

(73) Assignee: SHELL OIL COMPANY, Houston, TX (US)

(21) Appl. No.: 13/727,843

(22) Filed: Dec. 27, 2012

(30) Foreign Application Priority Data
Dec. 27, 2011 (EP) 11195818.7

(51) Int. Cl. C07C 1/22

(52) U.S. Cl. CPC C07C 1/22
USPC 585/315

(57) ABSTRACT

A process for the preparation of an olefin product, which process comprises the steps of: a) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene; b) separating at least a portion of the ethylene from the conversion effluent to form an ethylene stream; c) feeding the ethylene stream to an oligomerization step to produce higher molecular weight olefins; d) recycling at least a portion of the olefins as a recycle higher molecular weight olefins stream to step a).

This application claims the benefit of European Patent Application No. 11195818.7, filed Dec. 27, 2011, the entire disclosure of which is hereby incorporated by reference.

### FIELD OF THE INVENTION

The invention relates to an integrated process for preparing both lower olefins and higher olefins.

### BACKGROUND OF THE INVENTION

Oxygenate-to-olefin processes are well described in the art. Typically, oxygenate-to-olefin processes are used to produce predominantly ethylene and propylene. An example of such an oxygenate-to-olefin process is described in US Patent Application Publication No. 2011/112344, which is herein incorporated by reference. The publication describes a process for the preparation of an olefin product comprising ethylene and/or propylene, comprising a step of converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene.

The publication further describes possible integration with a cracker. The publication also describes partially hydrogenating a C₄ portion of the conversion effluent and/or cracker effluent and recycling at least part of the at least partially hydrogenated C₄ as recycle feedstock to the cracker or oxygenate-to-olefins conversion system.

Ethylene oligomerization processes are also described in the art. For example, U.S. Pat. No. 4,472,525 describes a process for oligomerizing ethylene to a mixture of olefinic products of high linearity in the presence of a catalyst comprising an atom of nickel in complex with an olefinically unsaturated compound and an o-dihydrocarbylphosphinophenyl alcohol or lower alkyl ether ligand. The olefins produced by this process are predominantly alpha olefins and comprise olefins in the plasticizer range, i.e., C₄-C₁₀; the detergent range, i.e., C₁₂-C₂₀; and higher olefins, e.g., polyethylene. Ethylene oligomerization processes have a fixed product slate that is determined by the catalyst used and the process conditions and it is not possible to make significant changes to the product slate as market conditions change.

### SUMMARY OF THE INVENTION

The invention provides a process for the preparation of an olefin product, which process comprises the steps of: a) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene; b) separating at least a portion of the ethylene from the conversion effluent to form an ethylene stream; c) feeding the ethylene stream to an oligomerization step to produce higher molecular weight olefins; and d) recycling at least a portion of the higher molecular weight olefins as a recycle olefin stream to step a).

### BRIEF DESCRIPTION OF THE DRAWINGS

FIG. 1 depicts an embodiment of a process flow scheme in accordance with the invention.

### DETAILED DESCRIPTION

“Higher molecular weight olefins” are defined herein as olefins having more than 2 carbon atoms. This includes propylene, butenes, pentenes, etc.

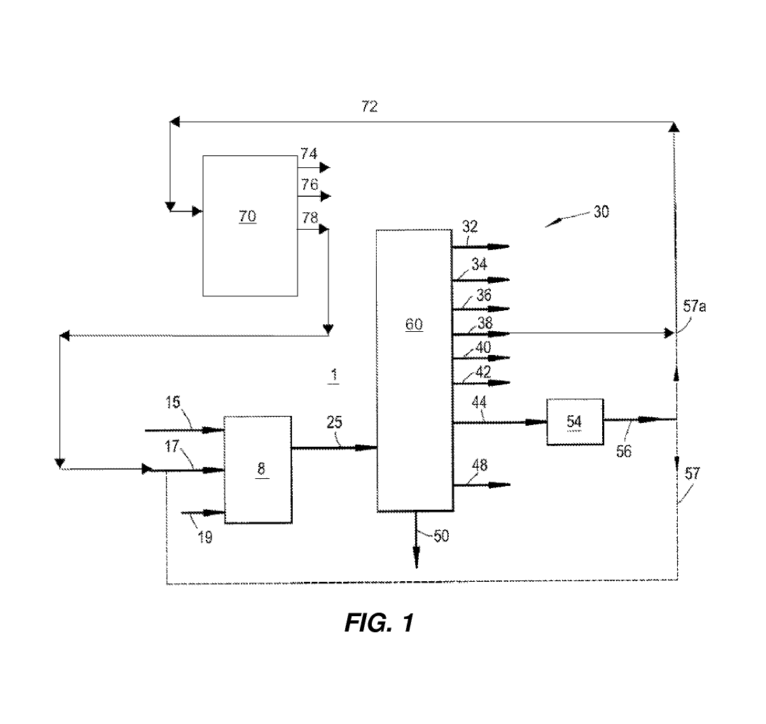

Reference is made to FIG. 1, showing an embodiment of a process flow scheme for an oxygenate-to-olefins conversion process.

The process comprises an oxygenate-to-olefins (OTO) conversion system 8, a higher olefin production section 70 and an OTO work-up section 60. An oxygenate feedstock is fed via line 15 to the OTO conversion system 8, for example, comprising methanol and/or dimethylether. Optionally, a hydrocarbon stream and/or a diluent are fed to the OTO conversion system via lines 17 or 19, respectively.

In principle every known OTO conversion system and process can be used in conjunction with the present invention, including processes known as Methanol-to-Olefins (MtO) and Methanol to Propylene (MtP). The OTO conversion system and process can for example be as disclosed in US 2005/0038304, incorporated herein by reference; as disclosed in US 2010/206771, incorporated herein by reference; or as disclosed in US 2006/020155 incorporated herein by reference. Other particularly suitable OTO conversion processes and systems with specific advantages are disclosed in US 2009/187058, US 2010/298619, US 2010/268009, US 2010/268007, US 2010/261943, and US 2011/160509, all of which are herein incorporated by reference.

In one embodiment, molecular sieve catalysts are used to convert oxygenate compounds to light olefins. Silicoaluminophosphate (SAPO) molecular sieve catalyst may be used that are selective to the formation of ethylene and propylene. Preferred SAPO catalysts are SAPO-17, SAPO-18, SAPO-34, SAPO-35, SAPO-44, the substituted forms thereof and mixtures thereof. The oxygenate feedstock may comprise one or more aliphatic containing compounds, including alcohols, amines, carbonyl compounds, for example, aldehydes, ketones and carboxylic acids, ethers, halides, mercaptans, sulfides, and the like and mixtures thereof. Examples of suitable feedstocks include methanol, ethanol, methyl mercaptan, ethyl mercaptan, methyl sulfide, methyl amine, di-methyl ether, di-ethyl ether, methyl ethyl ether, methyl chloride, ethyl chloride, dimethyl ketone, formaldehyde, acetaldehyde and various acids such as acetic acid.

In one embodiment, the oxygenate feedstock comprises one or more alcohols having from 1 to 4 carbon atoms and most preferably methanol. The oxygenate feedstock is contacted with a molecular sieve catalyst and is converted to light olefins, preferably ethylene and propylene.

Preferably, the OTO conversion system is arranged to receive an olefin stream, and is able to at least partially convert this stream, in particular a stream comprising C₄ olefins, to ethylene and/or propylene. In one embodiment, the olefin can be contacted with the oxygenate conversion catalyst in the OTO reaction zone; see for example, US 2009/187058, US 2010/298619 and US 2010/268009. The oxygenate conversion catalyst preferably comprises an aluminosilicate, in particular a zeolite.

In one embodiment, an olefinic co-feed is fed to the oxygenate-to-olefins conversion system. An olefinic co-feed is a feed containing one or more olefins or a mixture of olefins. The olefinic co-feed may also comprise other hydrocarbon compounds, for example, paraffinic compounds, alkylaromatic compounds, aromatic compounds or mixtures thereof. The olefinic co-feed preferably comprises more than 25 wt % olefins, more preferably more than 50 wt %, still more preferably more than 80 wt % and most preferably in the range of from 95 to 100 wt % olefins. A preferred olefinic co-feed consists essentially of olefins. Non-olefinic compounds in the olefinic co-feed are preferably paraffinic compounds.

The olefins in the olefinic co-feed are preferably mono-olefins. Further, the olefins can be linear, branched or cyclic, but they are preferably linear or branched. The olefins may have from 2 to 12 carbon atoms, preferably 3 to 10 carbon atoms and more preferably from 4 to 8 carbon atoms.

In another embodiment, the OTO conversion system comprises an olefin cracking zone downstream from the OTO reaction zone and is arranged to crack C₄₊ olefins produced in the OTO reaction zone, as described in U.S. Pat. No. 6,809,227 and US 2004/0102667, incorporated herein by reference. In one embodiment, the olefin produced in the OTO conversion system is fed to the olefin cracking zone.

In one embodiment, the yield of light olefins can be increased by converting the fraction that is heavier than propane to lighter olefins in an olefin cracking unit. The olefin cracking unit may use any molecular sieve catalyst capable of converting hydrocarbons with 4 or more carbon atoms into light olefins. Preferred molecular sieve catalysts for this olefin cracking unit are SAPO and zeolites as described hereinafter. The most preferred catalyst for this olefin cracking unit is ZSM-5.

Both the OTO process and the optional catalytic olefin cracking process may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

Catalysts suitable for converting the oxygenate feedstock preferably include molecular sieve-comprising catalyst compositions. Such molecular sieve-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing [TO₄] tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, 34, -35, -44, but also SAPO-5, -8, -11, -20, -31, -36, 37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIIB, VIIB, VIIIB and Lanthanide's of the Periodic Table of Elements, preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Aluminosilicate-comprising catalysts, and in particular zeolite-comprising catalysts, have the additional advantage that in addition to the conversion of methanol or ethanol, these catalysts also induce the conversion of olefins to ethylene and/or propylene. Furthermore, these aluminosilicate-comprising catalysts, and in particular zeolite-comprising catalysts, are particularly suitable for use as the catalyst in a catalytic olefin cracking zone. Particular preferred catalyst for this reaction, i.e. converting part of the olefins in the olefinic product, are catalysts comprising at least one zeolite selected from MFI, MEL, TON and MTT type zeolites, more preferably at least one of ZSM-5, ZSM-11, ZSM-22 and ZSM-23 zeolites.

In one preferred embodiment, the molecular sieve in the molecular sieve-comprising catalyst is a non-zeolitic molecular sieve, while part of the olefinic product, in particular at least part of the C4+ fraction containing olefins, is provided to a subsequent separate catalytic olefin cracking zone with a zeolite-comprising catalyst and the C4+ hydrocarbon fraction is at least partially converted by contact with the zeolite-comprising catalyst.

Preferred catalysts, for both the OTO reaction as well as an optional catalytic olefin cracking reaction, comprise a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

Particular catalysts, for both the OTO reaction as well as an optional olefin cracking reaction, include catalysts comprising one or more zeolite having one-dimensional 10-membered ring channels, i.e. one-dimensional 10-membered ring channels, which are not intersected by other channels. Preferred examples are zeolites of the MTT and/or TON type. Preferably, the catalyst comprises at least 40 wt %, preferably at least 50% wt of such zeolites based on total zeolites in the catalyst.

In a particularly preferred embodiment the catalyst, for both the OTO reaction as well as an optional catalytic olefin cracking reaction, comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst, for both the OTO reaction as well as an optional catalytic olefin cracking reaction, may comprise phosphorus as such or in a compound, i.e. phosphorus other than any phosphorus included in the framework of the molecular sieve. It is preferred that a MEL or MFI-type zeolites comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises MEL or MFI-type zeolites having SAR of in the range of from 60 to 150, more preferably of from 80 to 100, and phosphorus, wherein the phosphorus has preferably been introduced by post-treatment of the formulated catalyst. An even more particularly preferred catalyst comprises ZSM-5 having SAR of in the range of from 60 to 150, more preferably of from 80 to 100, and phosphorus, wherein the phosphorus has preferably been introduced by post-treatment of the formulated catalyst.

It is preferred that molecular sieves in the hydrogen form are used in the oxygenate conversion catalyst in step (g), e.g., HZSM-22, HZSM-23, and HZSM-48, HZSM-5. Preferably at least 50 wt %, more preferably at least 90 wt %, still more preferably at least 95 wt % and most preferably 100 w % of the total amount of molecular sieve used is in the hydrogen form. It is well known in the art how to produce such molecular sieves in the hydrogen form.

Typically the catalyst deactivates in the course of the process, primarily due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the molecular sieve. This applies to the catalyst for both the OTO reaction as well as an optional catalytic olefin cracking reaction.

The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

Suitable OTO processes will be further described in detail below. In the OTO conversion system 8, the oxygenate feedstock, and optionally an olefin co-feed (which can be partly or fully a recycle stream) are contacted with an oxygenate conversion catalyst under oxygenate conversion conditions, to obtain a conversion effluent comprising lower olefins in line 25. An optional diluent stream may comprise water, steam, inert gases such as nitrogen and/or paraffins, such as methane.

The reaction conditions of the oxygenate conversion include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Effluents from the OTO conversion system need to be worked up in order to separate and purify various components as desired, and in particular to separate one or more lower olefin product streams. FIG. 1 shows a work-up section 60 which receives and processes at least part of the conversion effluent.

Typically, the effluent is quenched in a quench unit with a quench medium such as water to cool the process gas before feeding it to a compressor. This allows for a smaller compressor and lower power consumption due to reduced gas volume. Any liquid hydrocarbons after the quench are phase separated from liquid water and separately recovered. The water or steam recovered from the quench unit can be partially recycled as diluent to the OTO conversion system via line 19. The water may be treated or purified, for example, to remove catalyst fines or to maintain the pH around neutral.

The vapor components after the quench are typically sent to a compression section, subjected to a caustic wash treatment, dried and sent to a separation including a cold section, to obtain separate streams of the main components. FIG. 1 shows hydrogen stream 32 which may contain some carbon monoxide, light ends stream 34 typically comprising methane and/or carbon monoxide, ethane stream 36, ethylene stream 38, propane stream 40, propylene stream 42, a C₄ stream 44, a C₅₊ stream 48 and a water effluent 50. There can also be a separate outlet for heavy (liquid) hydrocarbons. As known to one of ordinary skill in the art, the work-up section may be designed to provide different purities of each stream, and some of the streams will be produced from the work-up section as combined streams, i.e., C₄, C₅ and C₆ components can be combined. In a preferred embodiment, the C₅ and C₆ streams are combined. Additional reaction, treatment and/or purification steps may be carried out on any of these streams. For example, methane, carbon monoxide and hydrogen may be fed to a methanator to produce methane.

It may be advantageous to recycle at least part of the various streams from the workup section 60 to the OTO conversion system 8. Olefins having from 3 to 10 carbon atoms may be recycled to the OTO conversion system. In one embodiment, the heavier hydrocarbon streams may be fed to an olefin cracking zone as described above to produce additional light olefins. The heavier hydrocarbon streams may be those having more than 3 carbon atoms or preferably those having more than 4 carbon atoms, for example those having 5 and/or 6 carbon atoms.

FIG. 1 shows ethylene stream 38 being fed via line 72 to a higher olefin production process 70. The higher olefin production process is any process used to oligomerize ethylene including the Shell Higher Olefin Process which will be described in more detail herein, and a process using Ziegler catalysts to oligomerize ethylene. The SHOP process comprises an oligomerization step where the ethylene is oligomerized to form alpha olefins having at least 4 carbon atoms. The oligomerization process may be carried out according to the teachings of U.S. Pat. No. 4,472,525; U.S. Pat. No. 4,503,280; U.S. Pat. No. 4,472,522; and U.S. Pat. No. 4,503,279; all of which are herein incorporated by reference.

In one embodiment, the ethylene is fed in liquid phase solution in a reaction diluent into a reaction zone where it is contacted with a supported catalyst composition. The catalyst may be a nickel containing catalyst comprising nickel in complex with an olefinically unsaturated compound and a ligand, for example, an o-dihydrocarbylphosphinophenyl alcohol. During the oligomerization process, ethylene is converted to dimer, trimer, tetramer and like oligomers as well as polymers, i.e., polyethylene.

The ethylene oligomerization process generally produces alpha olefins having multiples of 2 carbon atoms, i.e., C₄, C₆, C₈, C₁₀, etc. An alpha olefin is defined as an olefin where the double bond is at the primary or alpha position. The product composition generally depends on the catalyst employed, the solvent employed, the reaction conditions and whether the catalyst is used in the heterogeneous or homogeneous state.

In one embodiment, some or all of the alpha olefins produced in the ethylene oligomerization step may be carried through an isomerization and disproportionation step. These steps produce internal olefins that can be used to produce alcohols and other derivatives for a variety of purposes. The isomerization and disproportionation steps may be carried out according to the teachings of U.S. Pat. No. 5,120,896; U.S. Pat. No. 5,043,520; U.S. Pat. No. 4,996,386; U.S. Pat. No. 4,895,997; and U.S. Pat. No. 4,727,203; all of which are herein incorporated by reference.

In one embodiment, olefins having from 4 to 100 carbon atoms are produced in the ethylene oligomerization step. The olefins are passed to a first separation zone where a product stream of detergent range olefins is separated from a light olefin stream having from 4 to 10 carbon atoms and a heavy olefin stream having from 20 to 100 carbon atoms. The light and heavy olefin streams are combined, optionally blended with additional olefins and fed to an isomerization step. In the isomerization step, the olefins are contacted with a catalyst under isomerization conditions to convert the alpha olefins to internal olefins. The internal olefins are then rearranged in a disproportionation step such that higher molecular weight olefins are reacted with lower molecular weight olefins to yield olefins of intermediate molecular weight. For example, a 15-triacontene (C₃₀H₆₀) is reacted with a 2-butene (C₄H₈) and disproportionate into two molecules of 2-heptadecene (C₁₇H₃₄). The isomerization and disproportionation steps produce additional detergent range alcohol as well as odd numbered carbon molecules, i.e., C₅, C₇, C₉, etc. The oligomerization step only produces even numbered carbon molecules, i.e., C₄, C₆, C₈, etc.

The integration of a higher olefin production process with an OTO conversion reactor allows for the product slate to be tailored to produce a subset of the olefins typically produced by the higher olefin production process. Undesired olefins may be recycled to the OTO conversion process via line 78 to convert them to ethylene and propylene that can be fed to the higher olefin production process or produced as products. The desired alpha olefin products from the oligomerization step can be separated and produced as products via line 74. An optional isomerization and disproportionation step can receive alpha olefins via line 76 that will be converted to internal olefins. The internal olefins can be hydroformylated to produce alcohols and/or further reacted to produce surfactant compositions useful in detergent and other applications.

A portion of the olefins from the isomerization and disproportionation steps can be recycled to the OTO conversion process. Internal olefins react similarly to alpha olefins in the OTO conversion system, so olefins from either the oligomerization step or the isomerization and disproportionation steps can be recycled to the OTO conversion system.

Alternatively, olefins produced in the isomerization step can be removed before being fed to the disproportionation step as either products or as additional olefins to be recycled to the OTO conversion system.

In an embodiment, any olefins having a carbon number less than or equal to 11 may be fed to the OTO process via line 78. This allows the integrated process to produce valuable olefins, preferably in the detergent range (C₁₂-C₂₀) without producing lower molecular weight olefins that may be more difficult to market and sell. Olefins having from 4 to 11 carbon atoms fed to the OTO process will be converted to ethylene and/or propylene. The ethylene can then be fed to the higher olefin production system to produce additional detergent range olefins. Alternatively, the ethylene and propylene can be produced as products from the workup section 60.

In an embodiment, olefins can be fed to an olefin cracking unit. Olefins having more than 4 carbon atoms can be fed to this type of unit and the olefins will be cracked into lower molecular weight olefins. For example, C₅ olefins will be cracked to ethylene and propylene.

In an embodiment, internal olefins produced in the isomerization and disproportionation step can be fed to the OTO conversion system as a recycle olefin stream or they can be fed to an olefin cracking unit.

In an embodiment a portion of the olefins can be fed to an olefin cracking unit and another portion of the olefins can be fed to the OTO conversion system. In one embodiment, the olefins having 4 or fewer carbon atoms are fed to the OTO conversion system and the olefins having 5 or more carbon atoms are fed to the olefin cracking unit. In another embodiment, the olefins having 5 or fewer carbon atoms are fed to the OTO conversion system and the olefins having 6 or more carbon atoms are fed to the olefin cracking unit.

For example, in one embodiment, the propylene can be produced as a product; the ethylene can be fed to the higher olefin production process; and the resulting products can be handled as follows: the C₄ olefins can be recycled to the OTO reactor; the C6 and C8 alpha olefins can be fed to the olefin cracking unit; the C₅, C₆, C₇, C₈ and C₉ internal olefins can be fed to the olefin cracking unit; and the detergent range olefins can be produced as products. In this embodiment, the lower molecular weight olefins will be recycled to produce additional detergent range olefins.

In another embodiment, one or more of the olefins having from 4 to 10 carbon atoms may be recycled to the OTO process. For example, if it is difficult to handle the amount of C₆ product produced by the higher olefin production process, the C₆ stream can be recycled while the C₈ stream is produced as a product and sold or converted to an alcohol or other derivative. The integration of the OTO process and the higher olefin production process provides a process that can produce a desired slate of products that can be shifted as dictated by market conditions.

In one embodiment, a portion of the olefins produced in the isomerization step can be separated from the olefin feed stream to the disproportionation step. By removing certain olefins, for example, the C₇ and C₉ internal olefins, the average number of carbon atoms can be changed resulting in a different product distribution from the disproportionation step. These olefins can be fed either to the OTO conversion system or the olefin cracking unit.

FIG. 1 shows the C₄ stream 44 being fed to a hydrogenation unit 54. All or part of the C₄ stream may be at least partially hydrogenated with a source of hydrogen. The at least partially dehydrogenated C4 stream can be recycled to the OTO conversion system via line 57 and line 17. When recycling to the OTO, the recycle C4 stream can be a co-feed to the OTO reaction zone or it can be a feed to an optional catalytic olefin cracking zone downstream from the OTO reaction zone. Suitable catalysts and conditions are described in U.S. Pat. No. 6,809,227 and US 2004/0102667. Catalysts include those comprising zeolite molecular sieves such as MFI-type, e.g., ZSM-5, or MEL-type, e.g., ZSM-11, as well as Boralite-D and silicalite 2.

In one particular embodiment, the stream 44 comprises a small quantity of di-olefins, in particular butadiene. A small quantity of butadiene is for example, at least 0.01 wt % of butadiene in the stream, in particular at least 0.1 wt %, more in particular at least 0.5 wt %. The stream comprising a small quantity of butadiene may be subjected to selective hydrogenation conditions in hydrogenation unit 54 to convert butadiene to butene, but preferably minimizing the hydrogenation of butene to butane. A suitable process for selective hydrogenation is described in U.S. Pat. No. 4,695,560. It is preferred for at least 90 wt % of the butadiene to be converted to butene and less than 10 wt %, preferably less than 5 wt % of the butene to be converted to butane. In another embodiment, the small quantity of butadiene may be left in the stream and recycled to the OTO conversion system.

The effluent from selective hydrogenation is a C₄ feedstock comprising butene, and butene is a desirable co-feed in OTO reactions, for example in a process in which a catalyst comprising an aluminosilicate or zeolite having one-dimensional 10-membered ring channels and an olefin co-feed is employed. The butene rich effluent can be recycled via line 57.

The olefin recycle stream 78 would preferably not pass through the hydrogenation section 54, because diolefins are not typically produced in the higher olefin production process. This will depend on the specific type of higher olefin production process used, but hydrogenation could be required for some streams depending on the diolefin levels or levels of other compounds.

In one embodiment, the olefins in the olefin recycle stream will be in a liquid form, and they would need to be heated and vaporized before being fed to the OTO conversion reactor.

In the OTO process and the ethylene oligomerization process, some paraffins are formed, for example C₄ saturates that will build up in the system until they are removed. An optional bleed line is present to remove these paraffins from the system. The bleed line may be used to purge saturates from the OTO process, the ethylene oligomerization process or a combined bleed line may be used for both processes. Valuable olefins are typically removed with this purge stream and it would be beneficial to reduce the amount of olefins purged from the system. The amount of olefins in the saturates bleed from the OTO process can be different from the amount of olefins in the saturates bleed from the ethylene oligomerization process. The streams may be combined. In one embodiment, the C₄ stream from the ethylene oligomerization step may be recycled to the OTO process via the recycle olefin stream and the saturates are only removed via a bleed line from the OTO process.

One method to reduce the amount of olefins purged from the system will be described herein. The C₄ stream in line 56 can optionally be used in a tert-alkyl ether step to produce one or more tert-alkyl ethers. The tert-alkyl ethers are a suitable oxygenate feed to the OTO conversion system, and the raffinate-2 stream can be fed to the olefin cracking process or to the optional isomerization and disproportionation steps. One embodiment of a tert-alkyl ether step comprises subjecting some or all of the C₄ stream to an etherification process with methanol and/or ethanol to produce a tert-alkyl ether. The iso-olefins contained in the C₄ stream react to form tert-alkyl ether and the remaining C₄ stream containing mostly linear butenes, known as raffinate-2, can be recycled or fed to one of the processes described above.

In one embodiment, some of the olefins via line 76 from the ethylene oligomerization step are fed to an olefin cracking unit. The olefins fed to the olefin cracking unit are preferably those having more than 4 carbon atoms. An optional isomerization and disproportionation step can be between the oligomerization step and the olefin cracking unit. The olefin cracking unit produces predominantly lower molecular weight olefins which are then fed to the workup section 60.

In one embodiment, the invention further comprises an isomerization and disproportionation step that receives an alpha olefin feed via line 76 from the oligomerization step. The isomerization and disproportionation step produces internal olefins, preferably in the detergent range that are exported as products or further reacted to produce detergent and/or other valuable components. Additional internal olefins can be recycled to the OTO conversion system.

### EXAMPLES

### Example 1

Three catalysts, comprising 40 wt % zeolite, 36 wt % kaolin and 24 wt % silica were tested to show their ability to convert 1-hexene to an olefinic product. To test the catalyst formulations for catalytic performance, the catalysts were pressed into tablets and the tablets were broken into pieces and sieved.

In the preparation of the first catalyst sample, ZSM-23 zeolite powder with a silica to alumina molar ratio (SAR) 46, and ZSM-5 zeolite powder with a SAR of 80 were used in the ammonium form in a weight ratio of 50:50. Prior to mixing the powders, the ZSM-5 zeolite powder was treated with phosphorus, resulting in a catalyst that has only one zeolite pre-treated with phosphorus. Phosphorus was deposited on a ZSM-5 zeolite powder with a silica-to-alumina ratio of 80 by means of impregnation with an acidic solution containing phosphoric acid to obtain a ZSM-5 treated zeolite powder containing 2.0 wt % P. The ZSM-5 powder was calcined at 550° C. Then, the powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution. Then, phosphorus was deposited on the catalyst by means of impregnation using acidic solutions containing phosphoric acid (H₃PO₄). The concentration of the solution was adjusted to impregnate 1.0 wt % of phosphorus on the weight of the formulated catalyst. After impregnation the catalysts were dried at 140° C. and were calcined at 550° C. for 2 hours. The final formulated catalyst thus obtained is further referred to as catalyst 1.

Another formulated catalyst was prepared as described herein above for catalyst 1, with the exceptions that ZSM-22 with a SAR of 100 was used instead of ZSM23. The final formulated catalyst thus obtained is further referred to as catalyst 2.

A third catalyst was prepared as described herein above for catalyst 1, with the exception that only ZSM-5 with a SAR of 80 was used and it was not treated with phosphorus prior to spray drying. The concentration of the phosphorus impregnation solution was adjusted to impregnate 1.5 wt % of phosphorus on the catalyst formulation. The final formulated catalyst thus obtained is further referred to as catalyst 3.

The phosphorus loading on the final catalysts is provided based on the weight percentage of the elemental phosphorus in any phosphor species, based on the total weight of the formulated catalyst.

1-Hexene, in the presence and absence of methanol, was reacted over the catalysts which were tested to determine their selectivity towards olefins, mainly ethylene and propylene. For the catalytic testing, a sieve fraction of 60-80 mesh was used. The reaction was performed using a quartz reactor tube of 1.8 mm internal diameter. The molecular sieve samples were heated in nitrogen to the reaction temperature and a mixture consisting of 3 vol % 1-hexene, 6 vol % methanol balanced in N₂ was passed over the catalyst at atmospheric pressure (1 bar). In another experiment 3 vol % of 1-hexene balanced in N₂ was passed over the catalyst at atmospheric pressure (1 bar).

The Gas Hourly Space Velocity (GHSV) is determined by the total gas flow over the zeolite weight per unit time (ml gas)/(g zeolite·hr). The gas hourly space velocity used in the experiments was 19,000 (ml/(g·hr)). The temperature used in the experiments was 525° C. The effluent from the reactor was analyzed by gas chromatography (GC) to determine the product composition. The composition was calculated on a weight basis of all hydrocarbons analyzed. The composition has been defined by the division of the mass of specific product by the sum of the masses of all products. The effluent from the reactor obtained at several reactor temperatures was analyzed. The results are shown in Table 1. As can be seen from the results, the main product is propylene, and smaller amounts of ethylene and C4 compounds are also formed.

TABLE 1

| | 1-Hexene | MeOH | C2= | C3= | C4 | C5 | C6+ | LE | C4 sat/C4 |
|---|---|---|---|---|---|---|---|---|---|
| Catalyst | (vol %) | (vol %) | wt % | wt % | wt % | wt % | wt % | wt % | total wt/wt |
| 1 | 3 | 0 | 9.43 | 69.01 | 16.46 | 1.70 | 3.27 | 0.12 | 0.84 |
| 1 | 3 | 6 | 12.94 | 54.56 | 23.85 | 3.02 | 5.22 | 0.40 | 2.70 |
| 2 | 3 | 0 | 11.23 | 64.65 | 17.99 | 2.03 | 3.84 | 0.27 | 1.24 |
| 2 | 3 | 6 | 14.49 | 52.84 | 23.22 | 3.22 | 5.97 | 0.26 | 3.87 |
| 3 | 3 | 0 | 12.87 | 61.97 | 18.84 | 2.11 | 4.15 | 0.06 | 1.72 |
| 3 | 3 | 6 | 15.89 | 52.55 | 22.54 | 3.02 | 5.90 | 0.10 | 4.30 |

### Example 2

Example 2 was carried out in a similar manner to Example 1, except that 1-octene instead of 1-hexene, in the presence and absence of methanol, was reacted over the catalysts which were tested to determine their selectivity towards olefins. The results are shown in Table 2. As can be seen from the results, the main product is propylene, and smaller amounts of ethylene and C4 compounds are also formed.

TABLE 2

| | 1-Octene | MeOH | C2= | C3= | C4 | C5 | C6+ | LE | C4 sat/C4 |
|---|---|---|---|---|---|---|---|---|---|
| Catalyst | (vol %) | (vol %) | wt % | wt % | wt % | wt % | wt % | wt % | total wt/wt |
| 1 | 3 | 0 | 17.51 | 47.19 | 26.06 | 3.63 | 5.51 | 0.09 | 5.18 |
| 1 | 3 | 6 | 17.10 | 47.83 | 23.56 | 4.20 | 7.08 | 0.23 | 6.48 |
| 2 | 3 | 0 | 14.78 | 45.77 | 30.34 | 4.36 | 4.60 | 0.15 | 3.12 |
| 2 | 3 | 6 | 13.48 | 49.55 | 26.32 | 4.49 | 6.01 | 0.16 | 3.97 |
| 3 | 3 | 0 | 16.10 | 46.73 | 27.80 | 3.92 | 5.29 | 0.16 | 4.59 |
| 3 | 3 | 6 | 15.16 | 48.64 | 24.79 | 4.27 | 6.92 | 0.21 | 5.47 |

### Example 3

Example 3 was carried out in a similar manner to Example 1, except that 1-butene instead of 1-hexene, in the presence and absence of methanol, was reacted over the catalysts which were tested to determine their selectivity towards olefins. The results are shown in Table 3. As can be seen from the results, the main product is propylene when it is co-fed with methanol, and smaller amounts of ethylene and C4 compounds are also formed.

TABLE 3

| | 1-Butene | MeOH | C2= | C3= | C4 | C5 | C6+ | LE | C4 sat/C4 |
|---|---|---|---|---|---|---|---|---|---|
| Catalyst | (vol %) | (vol %) | wt % | wt % | wt % | wt % | wt % | wt % | total wt/wt |
| 1 | 3 | 0 | 4.23 | 13.57 | 77.54 | 0.58 | 0.83 | 3.23 | 1.11 |
| 1 | 3 | 6 | 13.59 | 50.66 | 27.37 | 2.47 | 4.31 | 1.60 | 3.70 |
| 3 | 3 | 0 | 8.00 | 23.74 | 63.30 | 0.77 | 1.91 | 2.27 | 2.07 |
| 3 | 3 | 6 | 16.66 | 50.05 | 23.75 | 2.41 | 5.78 | 1.36 | 5.15 |

What is claimed is:

1. A process for the preparation of an olefin product, which process comprises the steps of:
a. converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene;
b. separating at least a portion of the ethylene from the conversion effluent to form an ethylene stream;
c. feeding the ethylene stream to an oligomerization step to produce higher molecular weight olefins;
d. recycling at least a portion of the olefins as a recycle higher molecular weight olefins stream to step a).

2. A process as claimed in claim 1 wherein the recycle higher molecular weight olefins stream is contacted with the oxygenate feedstock and the oxygenate conversion catalyst.

3. A process as claimed in claim 1 wherein the recycle higher molecular weight olefins stream comprises olefins having from 4-10 carbon atoms.

4. A process as claimed in claim 1 wherein the recycle higher molecular weight olefins stream comprises at least 50% alpha olefins.

5. A process as claimed in claim 1 wherein additional olefins other than the recycle higher molecular weight olefins stream are fed to step a) with the oxygenate feedstock.

6. A process as claimed in claim 5 wherein the additional olefins comprise olefins having from 6 to 10 carbon atoms.

7. A process as claimed in claim 1 wherein the oxygenate feedstock is selected from the group consisting of methanol, ethanol, tert-alkyl ethers and mixtures thereof.

8. A process as claimed in claim 1 further comprising a step of isomerizing and disproportionating at least a portion of the olefins produced in step c) to produce internal olefins.

9. A process as claimed in claim 8 further comprising recycling at least a portion of the internal olefins to step a).

10. A process as claimed in claim 1 wherein the recycle olefin stream comprises at least 50% internal olefins.

11. A process as claimed in claim 8 further comprising converting at least a portion of the internal olefins to alcohols and/or alcohol derivatives.

12. A process as claimed in claim 10 wherein the recycle olefin stream comprises olefins having from 6 to 10 carbon atoms.

13. A process as claimed in claim 1 further comprising contacting at least a portion of the olefins produced in step c) with a catalyst in an olefin cracking zone to produce a cracking effluent.

14. A process as claimed in claim 10 wherein the olefins contacted in the olefin cracking zone comprise olefins having from 5 to 10 carbon atoms.

15. A process as claimed in claim 8 further comprising a tert-alkyl ether production step wherein at least a portion of the conversion effluent from step a) is contacted with methanol and/or ethanol under tert-alkyl ether production conditions to produce a tert-alkyl ether and a raffinate-2 stream.

16. A process as claimed in claim 15 wherein the portion of the conversion effluent and the raffinate-2 stream comprise predominantly olefins having four carbon atoms.

17. A process as claimed in claim 15 further comprising feeding at least a portion of the tert-alkyl ether to the oxygenate-to-olefins conversion system of step a).

18. A process as claimed in claim 15 further comprising feeding at least a portion of the raffinate-2 to the isomerization and disproportionation step.
